(19) United States
(12) Patent Application Publication
(10) Pub. No.: US 2014/0039319 A1
(43) Pub. Date: Feb. 6, 2014

(54) ULTRASONIC MEASURING APPARATUS AND BLOOD VESSEL INNER DIAMETER CALCULATING METHOD

(71) Applicant: SEIKO EPSON CORPORATION, Tokyo (JP)

(72) Inventor: Tomonori MANO, Matsumoto-shi (JP)

(21) Appl. No.: 13/958,003

(22) Filed: Aug. 2, 2013

(30) Foreign Application Priority Data
Aug. 6, 2012 (JP) 2012-173751
Aug. 2, 2013 (JP) 2013-161074

(51) Int. Cl. A61B 8/08

(52) U.S. Cl. CPC A61B 8/0891; A61B 8/5223
USPC 600/449

(57) ABSTRACT

In an ultrasonic measuring apparatus, an addition/averaging-period setting unit determines a blood vessel diameter stable period in which a blood vessel diameter is in a stable state in one heartbeat period and sets the blood vessel diameter stable period as an addition and averaging period. An adding and averaging unit adds up and averages reflected wave measurement data measured in the addition and averaging period set by the addition/averaging-period setting unit. A blood-vessel-diameter calculating unit calculates a blood vessel inner diameter of a blood vessel using combined data obtained by the addition and averaging of the adding and averaging unit.

### BACKGROUND

1. Technical Field

The present invention relates to an ultrasonic measuring apparatus and the like for measuring a blood vessel inner diameter.

2. Related Art

There have been devised an apparatus for measuring a blood flow, a blood vessel diameter, and a blood pressure using ultrasound and an apparatus for measuring a modulus of elasticity of a blood vessel. These apparatuses can perform noninvasive measurement without causing pain and discomfort of a subject. For example, JP-A-2006-51285 (Patent Literature 1) discloses a technique for measuring a blood vessel diameter using a reflected wave of ultrasound emitted onto a measurement target blood vessel.

When the blood vessel diameter of the measurement target blood vessel is measured, an ultrasonic beam is emitted vertically with respect to the major axis of the measurement target blood vessel and a reflected wave of the ultrasonic beam is detected to perform the measurement. A blood vessel wall roughly includes the intima, the media, and the exine. An inter-exine distance is often used as the blood vessel diameter. However, because of the structure of the exine, a reflected wave from the exine includes reflection components from a plurality of reflection positions between the exine and the media. Therefore, high blood vessel diameter accuracy cannot be obtained.

For example, when it is assumed that a blood pressure is estimated using a correlation characteristic of a blood vessel diameter and a blood pressure, accuracy of about 20 to 30 μm is necessary as measurement accuracy for the blood vessel diameter. To obtain the accuracy, the inter-exine distance is insufficient. It is necessary to measure a blood vessel inner diameter. To measure the blood vessel inner diameter, it is necessary to calculate a distance between lumen-intima boundaries. However, reflected waves from the lumen-intima boundaries are relatively small compared with a reflected wave from the exine and tend to be buried under noise. Therefore, it is difficult to highly accurately measure a blood vessel inner diameter.

Patent Literature 1 discloses a method of reducing the influence of noise such as multiple reflection and highly accurately calculating an inter-exine distance. However, Patent Literature 1 does not disclose a method of correctly detecting the lumen-intima boundaries to highly accurately calculate a blood vessel inner diameter.

### SUMMARY

An advantage of some aspects of the invention is to propose a new method for correctly measuring a blood vessel inner diameter.

A first aspect of the invention is directed to an ultrasonic measuring apparatus that executes emission of ultrasound and measurement of a reflected wave from a blood vessel and detects fluctuation in a blood vessel diameter using measurement data, the ultrasonic measurement apparatus including: a determining unit configured to determine, on the basis of the measurement data, a blood vessel diameter stable period in which the blood vessel diameter is in a stable state in one heartbeat period; a combining unit configured to combine the measurement data measured in the blood vessel diameter stable period; and a blood-vessel-inner-diameter calculating unit configured to calculate a blood vessel inner diameter of the blood vessel using the data combined by the combining unit.

As another aspect, the invention may be configured as a blood vessel inner diameter calculating method for executing emission of ultrasound and measurement of a reflected wave from a blood vessel and detecting fluctuation in a blood vessel diameter using measurement data, the blood vessel inner diameter calculating method including: determining, on the basis of the measurement data, a blood vessel diameter stable period in which the blood vessel diameter is in a stable state in one heartbeat period; combining the measurement data measured in the blood vessel diameter stable period; and calculating a blood vessel inner diameter of the blood vessel using the combined data.

According to the first aspect and the like of the invention, a blood vessel diameter stable period in which a blood vessel diameter is in a stable state in one heartbeat period is determined on the basis of a result of detection of fluctuation in the blood vessel diameter performed using measurement data obtained by executing emission of ultrasound and measurement of a reflected wave from a blood vessel. The measurement data measured in the blood vessel diameter stable period is combined. In the blood vessel diameter stable period, the position of a blood vessel wall is also stable. By combining a plurality of measurement data in the blood vessel diameter stable period, it is possible to reduce a noise component and relatively clarify a signal component. That is, a peak of a reflected wave from a lumen-intima boundary necessary for calculating a blood vessel inner diameter becomes clear and accuracy of peak detection is improved. Therefore it is possible to correctly calculate the blood vessel inner diameter of the blood vessel using the combined data.

As a second aspect, the ultrasonic measuring apparatus according to the first aspect of the invention may be configured such that the combining unit adds up and averages the measurement data measured in the blood vessel diameter stable period.

According to the second aspect of the invention, the measurement data measured in the blood vessel diameter stable period is added up and averaged. Consequently, it is possible to effectively reduce a noise component in the measurement data and clarify a peak of a reflected wave from a lumen-intima boundary.

As a third aspect, the ultrasonic measuring apparatus according to the first or second aspect of the invention may be configured such that the determining unit determines the blood vessel diameter stable period from a diastole.

In the diastole, there is a period in which the blood vessel diameter is stabilized. Therefore, in the third aspect of the invention, the blood vessel diameter stable period is determined from the diastole. Consequently, it is possible to easily select a period suitable for the combination of the measurement data.

As a fourth aspect, the ultrasonic measuring apparatus according to any of the first to third aspect of the invention may be configured such that the blood-vessel-inner-diameter calculating unit includes a peak detecting unit configured to detect a reflected wave peak of the lumen-intima boundary from the data combined by the combining unit and calculates the blood vessel inner diameter on the basis of the reflected wave peak.

According to the fourth aspect of the invention, a reflected wave peak of the lumen-intima boundary is detected from the data combined by the combining unit. It is possible to appropriately calculate a blood vessel inner diameter on the basis of the reflected wave peak.

As a fifth aspect of the invention, the ultrasonic measuring apparatus according to the fourth aspect of the invention may be configured such that the ultrasonic measuring apparatus further includes a range setting unit configured to set, on the basis of the measurement data, a depth range in which the lumen-intima boundary can be present in the blood vessel diameter stable period, and the peak detecting unit detects the reflected wave peak using the depth range.

The position of the lumen-intima boundary fluctuates according to contraction and expansion of the blood vessel. Therefore, the depth range in which the lumen-intima boundary can be present changes according to the point in a cycle for expansion and contraction of the blood vessel the blood vessel diameter stable period is. Therefore, according to the fifth aspect of the invention, a depth range in which the lumen-intima boundary can be present in the blood vessel diameter stable period is set on the basis of the measurement data. A reflected wave peak is detected using the set depth range. Consequently, it is possible to improve accuracy of peak detection for a reflected wave from the lumen-intima boundary.

As a sixth aspect, the ultrasonic measuring apparatus according to the fourth or fifth aspect of the invention may be configured such that the ultrasonic measuring apparatus further includes a blood-vessel-inner-diameter-fluctuation calculating unit configured to set a position in the measurement data of the reflected wave peak detected by the peak detecting unit as a tracking target, track the position of the reflected wave peak in the continuous measurement data, and calculate fluctuation in the blood vessel inner diameter.

According to the sixth aspect of the invention, a position in the measurement data of the reflected wave peak detected by the peak detecting unit is set as a track target and positions of the reflected wave peak in the continuous measurement data are tracked. Consequently, it is possible to correctly calculate fluctuation in the blood vessel inner diameter.

### BRIEF DESCRIPTION OF THE DRAWINGS

The invention will be described with reference to the accompanying drawings, wherein like numbers reference like elements.

FIG. 1A is a schematic configuration diagram of an ultrasonic measuring apparatus.

FIG. 1B is an explanatory diagram of a lumen-intima boundary.

FIG. 2A is a diagram showing an example of measurement data.

FIG. 23 is a diagram showing an example of combined data.

FIG. 3A is a diagram showing an example of blood vessel diameter fluctuation in one heartbeat period.

FIG. 3B is a partially enlarged view of a diastole end.

FIG. 4 is a block diagram showing an example of a functional configuration of the ultrasonic measuring apparatus.

FIG. 5 is a diagram showing an example of a data configuration of reference measurement data.

FIG. 6 is a diagram showing an example of a data configuration of blood vessel inner diameter measurement data.

FIG. 7 is a flowchart for explaining a flow of blood vessel inner diameter measurement processing.

FIG. 8 is a diagram showing an example of blood vessel diameter fluctuation in one heartbeat period.

FIG. 9 is a flowchart showing a part of steps extracted from second blood vessel inner diameter measurement processing,

### DESCRIPTION OF EXEMPLARY EMBODIMENT

An example of a preferred embodiment applied with the invention is explained below with reference to the drawings. However, it goes without saying that forms applicable with the invention are not limited to the embodiment explained below.

### 1. Apparatus Configuration

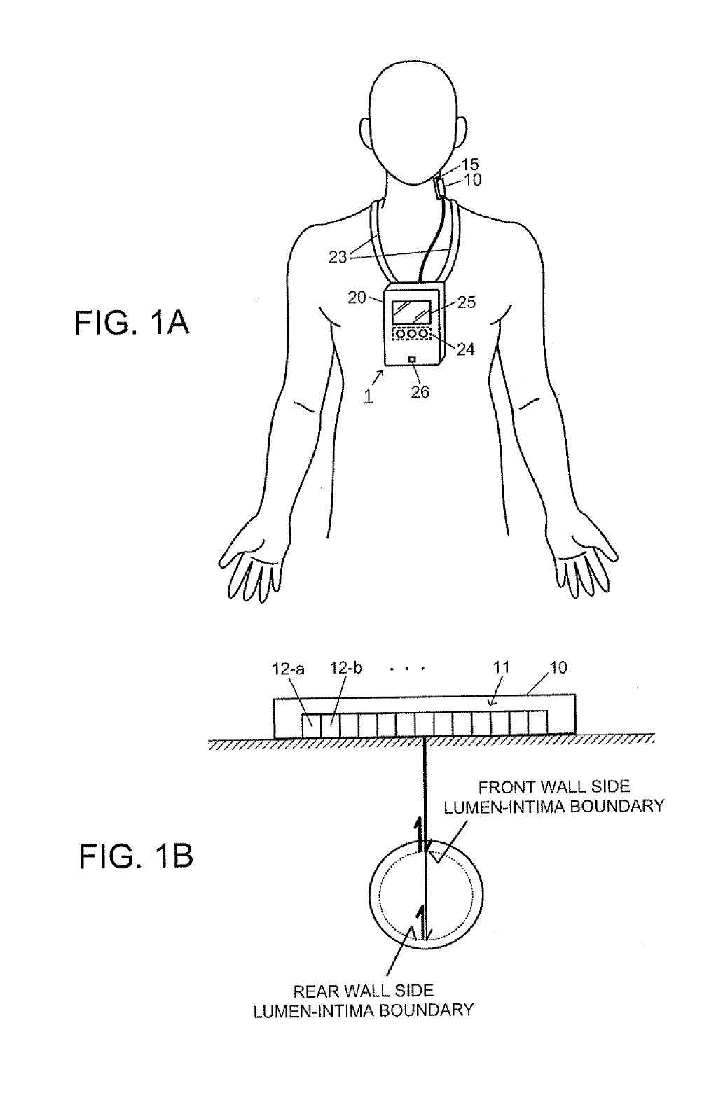

FIG. 1A is a schematic configuration diagram of an ultrasonic measuring apparatus 1 in this embodiment. The ultrasonic measuring apparatus 1 includes an ultrasonic probe 10 and a main body apparatus 20. A subject wears the ultrasonic measuring apparatus 1 using a bonding tape 15 to locate the ultrasonic probe 10 on the carotid, sets the carotid as a measurement target blood vessel, and measures a blood vessel inner diameter of the carotid. The ultrasonic measurement apparatus 1 can also be considered a blood vessel inner diameter measuring apparatus for measuring a blood vessel inner diameter.

The ultrasonic probe 10 transmits a pulse signal or a burst signal of ultrasound of several megahertz to several ten megahertz from a transmitting unit to the carotid. The ultrasonic probe 10 receives a reflected wave from the carotid in a receiving unit and outputs a reception signal of the reflected wave to the main body apparatus 20.

The main body apparatus 20 is an apparatus main body of the ultrasonic measuring apparatus 1. The main body apparatus 20 is wire-connected to the ultrasonic probe 10 via a cable. A neck strap 23 used by the subject to hang the main body apparatus 20 from the neck is attached to the main body apparatus 20.

Operation buttons 24, a liquid crystal display device 25, and a speaker 26 are provided on the front surface of the main body apparatus 20. Although not shown in the figure, a control board for comprehensively controlling the apparatus is built in the main body apparatus 20. A microprocessor, a memory, a circuit for transmission and reception of ultrasound, a battery, and the like are mounted on the control board.

The operation buttons 24 is used by the subject to input a measurement start instruction for a blood vessel inner diameter and various amounts related to the measurement of the blood vessel inner diameter.

A measurement result of the blood vessel inner diameter by the ultrasonic measuring apparatus 1 is displayed on the liquid crystal display device 25. As a display method, a measurement value of the blood vessel inner diameter may be displayed as a numerical value or may be displayed as a graph or the like.

Various kinds of sound guidance and the like related to the measurement of the blood vessel inner diameter are output from the speaker 26.

In the ultrasonic probe 10, ultrasonic transducer arrays 11 including a plurality of ultrasonic transducers 12 (12-a, 12-b, etc.) for transmitting and receiving ultrasound are linearly arranged. The ultrasonic probe 10 is configured to be capable of switching the ultrasonic transducer array 11 for transmitting an ultrasonic beam, changing a transmitting direction of the ultrasonic beam to be transmitted, and changing a so-called focus position. Since control itself of these devices is publicly known, detailed explanation of the control is omitted.

### 2. Principle

FIG. 1B is a cross sectional view of the neck schematically showing a positional relation between the ultrasonic probe 10 and the measurement target blood vessel. The figure is focused on one ultrasonic transducer array 11. The blood vessel includes the lumen, the intima, the media, and the exine. However, the media is not shown for simplification of illustration.

An ultrasonic beam (a scanning line) is formed by controlling transmission of ultrasound from the ultrasonic transducers included in the ultrasonic transducer array 11. In this figure, the ultrasonic beam is transmitted from the center of the ultrasonic transducer array to the measurement target blood vessel (in this embodiment, the carotid).

The ultrasonic beam has a characteristic that the ultrasonic beam is reflected in a portion where a difference in impedance is present. The ultrasonic beam transmitted through the exine travels from the media to the intima and is reflected on a boundary between the intima and the lumen (hereinafter referred to as “lumen-intima boundary”). In this embodiment, measurement data of a reflected wave of the ultrasonic beam is continuously acquired in time series and a blood vessel inner diameter is measured using the obtained measurement data.

In the lumen-intima boundary, lumen-intima boundaries respectively on a front wall side and a rear wall side are present viewed from the ultrasonic probe 10. In this embodiment, the lumen-intima boundary on the front wall side is referred to as “front wall side lumen-intima boundary” and the lumen-intima boundary on the rear wall side is referred to as “rear wall side lumen-intima boundary”. The ultrasonic beam is reflected on the front wall side lumen-intima boundary and the rear wall side lumen-intima boundary. Reflected waves of the ultrasonic beam are received (detected) by the ultrasonic transducers.

Although not shown in FIG. 1B, the ultrasound is largely reflected on a boundary between the media and the exine (hereinafter referred to as “media-exine boundary”). In this embodiment, the media-exine boundary on the front wall side is referred to as “front wall side media-exine boundary” and the media-exine boundary on the rear wall side is referred to as “rear wall side media-exine boundary”.

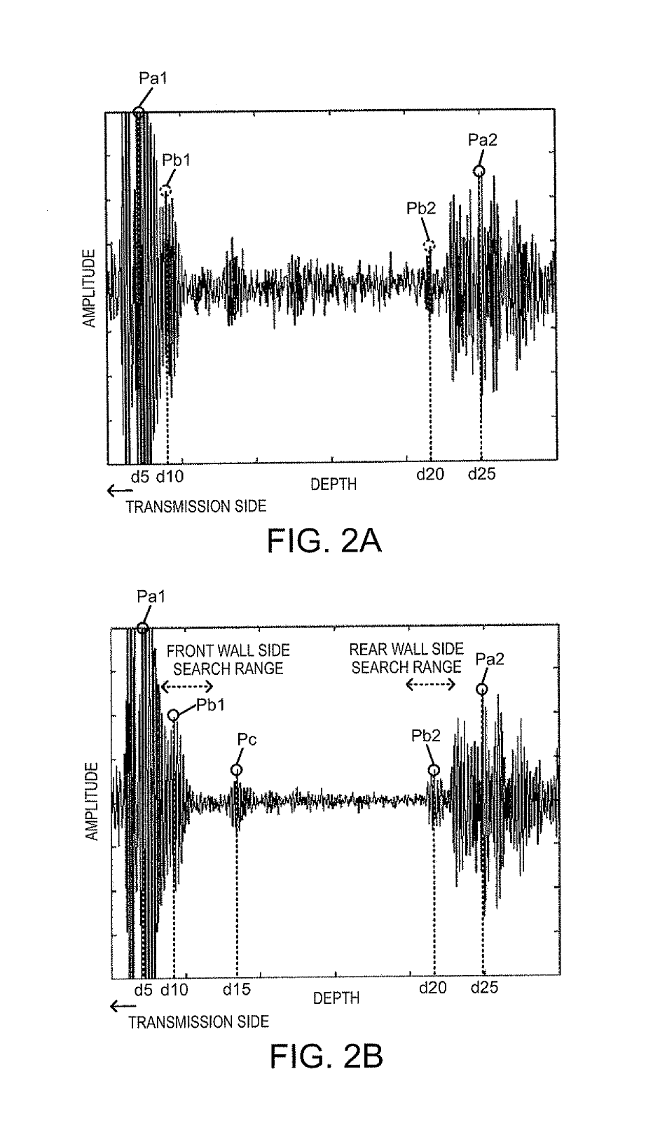

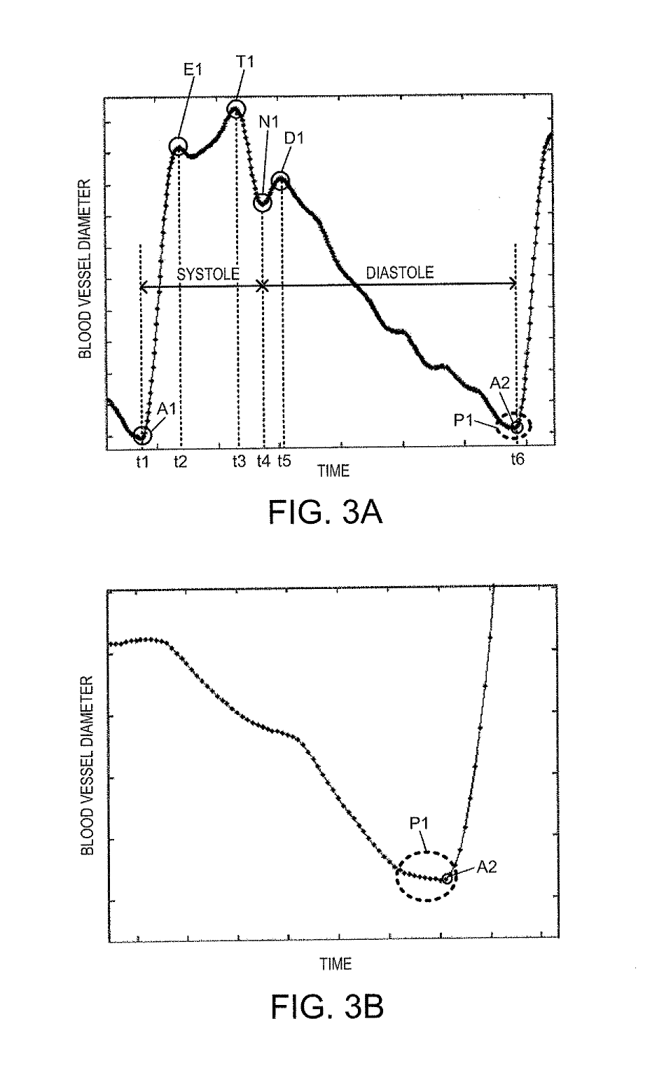

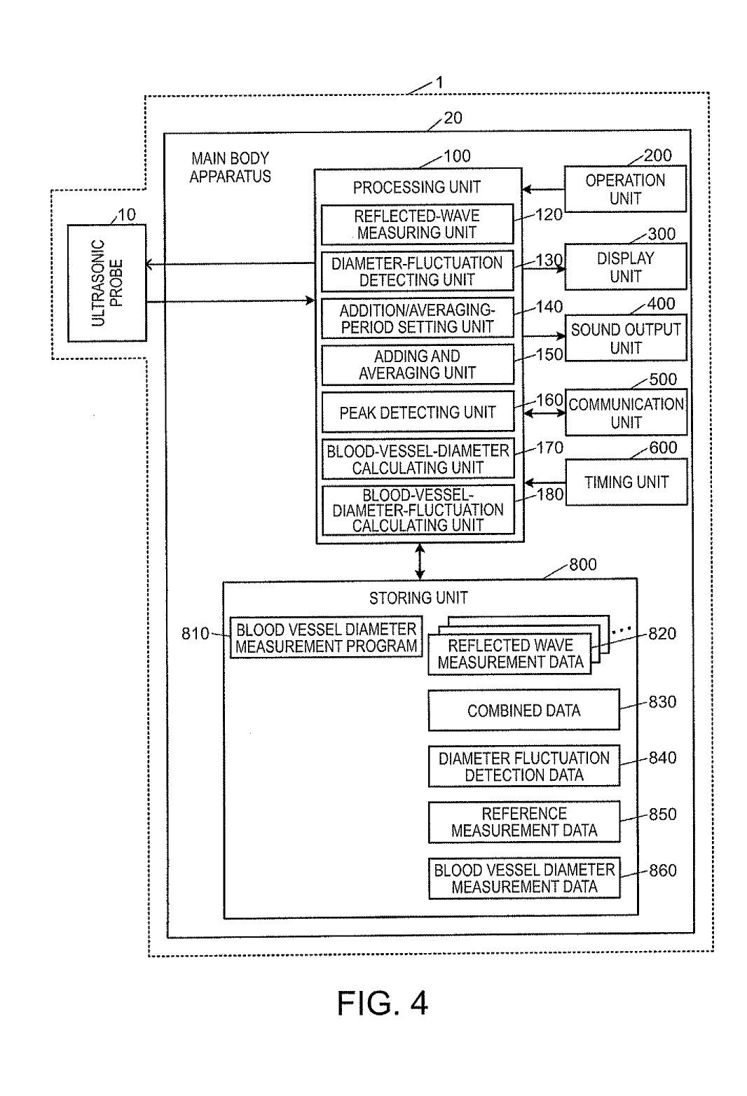

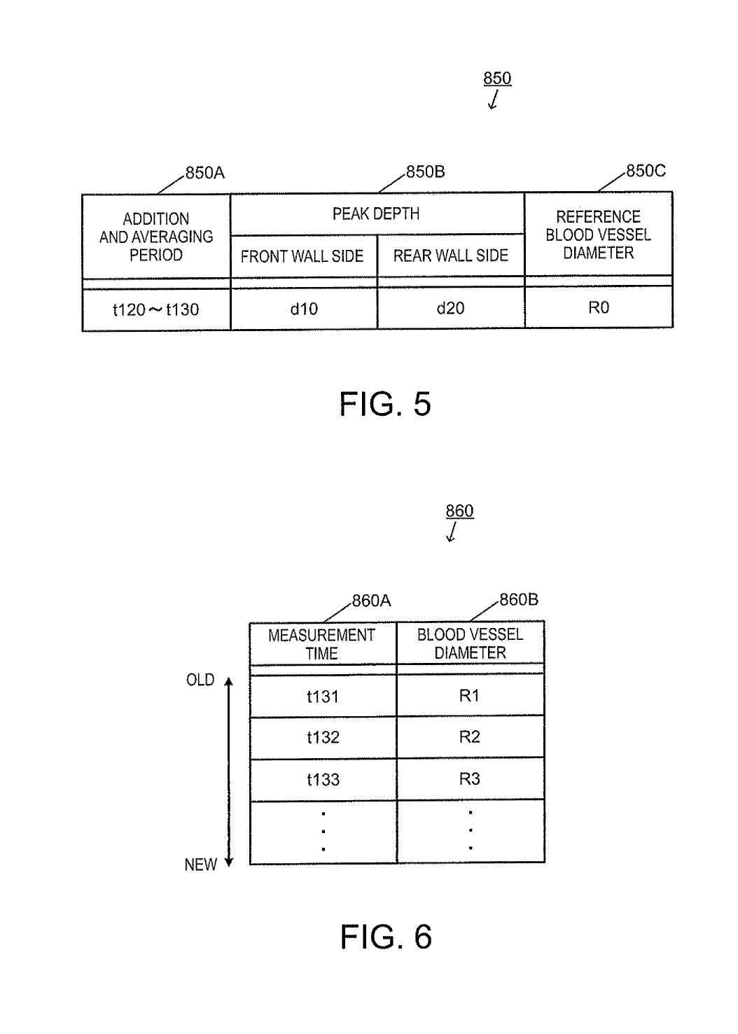

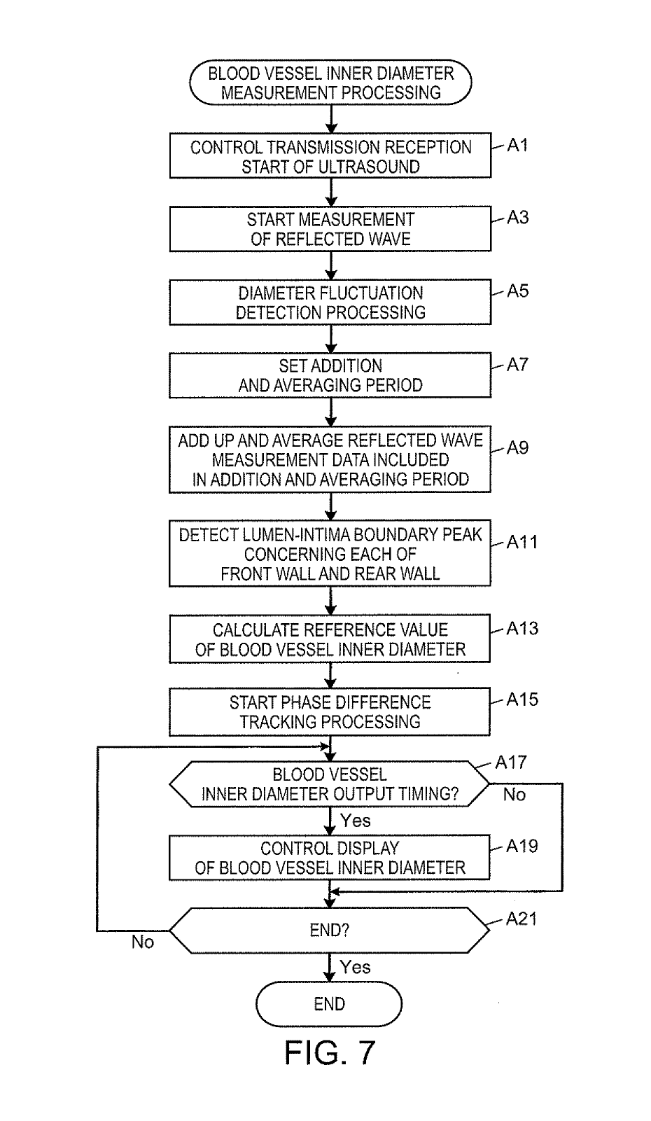

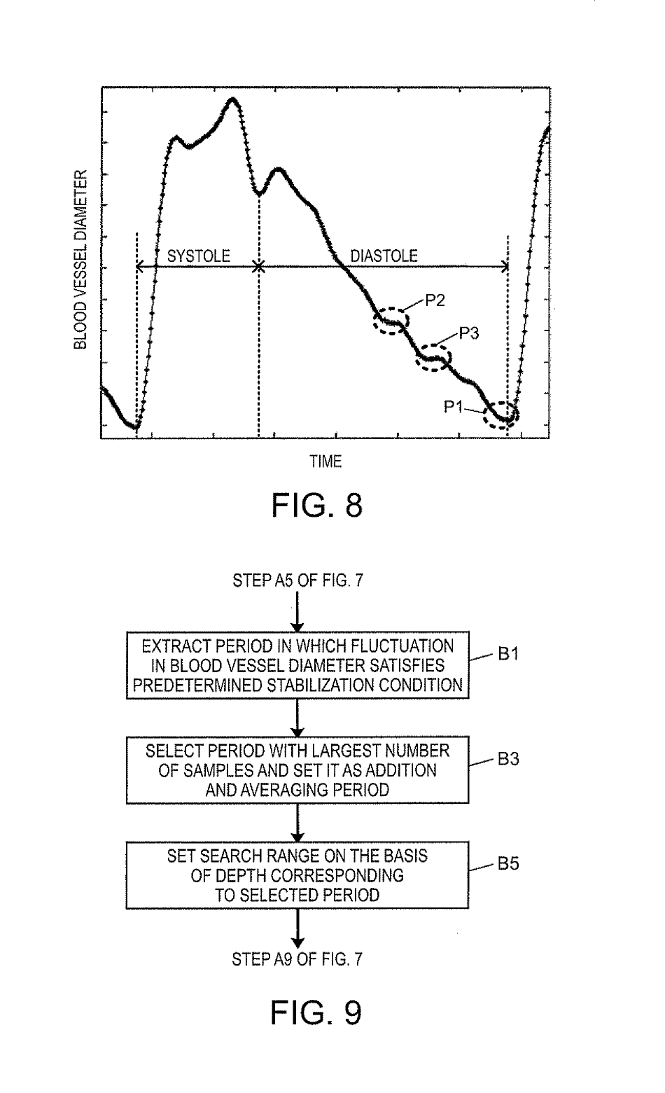

FIG. 2A is a diagram showing, with respect to depth in a living organism, an example of a result obtained by converting the intensity of a reflected wave received by the ultrasonic probe 10 into amplitude. The left side in the figure is an ultrasound transmission side (a probe side). The abscissa indicates the depth and the ordinate indicates the amplitude. This data is measurement data of the reflected wave in one measurement. About several ten to several hundred measurement data are obtained in one second. When one measurement is defined as one frame, the measurement data can also be considered frame data.

It is seen from the figure that peak groups having large peaks appear on the transmission side of the ultrasound. Among the peak groups, a peak Pa1 observed at depth d5 is a peak equivalent to the front wall side media-exine boundary (hereinafter referred to as “front wall side media-exine boundary peak”).

A peak Pb1 lower than the peak Pa1 is observed at depth d10 slightly larger than the depth d5. The peak Pb1 is a peak equivalent to the front wall side lumen-intima boundary (hereinafter referred to as “front wall side lumen-intima boundary peak”).

Reflection of the ultrasound hardly occurs in a lumen portion of the measurement target blood vessel. Therefore, the amplitude of the reflected wave is relatively small in a range of depth d10 to depth d20. At the depth d20, a slightly high peak Pb2 is observed. The peak Pb2 is a peak equivalent to the rear wall side lumen-intima boundary (hereinafter referred to as “rear wall side lumen-intima boundary peak”). Large peak groups are observed again in a region deeper than the depth d20. Among the peak groups, a peak Pat observed at depth d25 is a peak equivalent to the rear wall side media-exine boundary (hereinafter referred to as “rear wall side media-exine boundary peak”).

A peak on the rear wall side has relatively smaller amplitude than a peak on the front wall side. This is because, as a distance from a transmission position of the ultrasound increases, an ultrasonic signal is attenuated, the intensity of the ultrasonic signal is weakened, and a reflected wave of the ultrasonic signal is also attenuated while propagating to the transmission position.

In this way, when the ultrasonic beam is irradiated on the measurement target blood vessel, it is seen that there are four peaks, i.e., the front wall side media-exine boundary peak Pa1, the front wall side lumen-intima boundary peak Pb1, the rear wall side lumen-intima boundary peak Pb2, and the rear wall side media-exine boundary peak Pat in order from the one at the smallest depth.

However, among the four peaks, in particular, in a rear wall side lumen-intima boundary peak Pb2, it is difficult to distinguish the position of the peak and specify an accurate position of the peak. Nevertheless, FIG. 2A is relatively easy to see. However, in general, the amplitude of the rear wall side lumen-intima boundary peak Pb2 is a level buried under noise. Therefore, if it is attempted to detect the lumen-intima boundary peak (in particular, the rear wall side lumen-intima boundary peak) from the measurement data itself of the reflected wave shown in FIG. 2A, likelihood of misdetection is high. Therefore, in this embodiment, the lumen-intima boundary peak is detected according to a procedure explained below and a blood vessel inner diameter of the measurement target blood vessel is calculated using a result of the detection.

### 2-1. Detection of Blood Vessel Diameter Fluctuation

First, emission of ultrasound and measurement of a reflected wave from a blood vessel by the ultrasonic probe 10 are repeatedly executed. Fluctuation in a blood vessel diameter is detected using measurement data of the measurement by a diameter fluctuation detection unit 130 in a main body apparatus 20 described later. From the measurement data shown in FIG. 2A, the media-exine boundary peaks are clear and it is easy to specify the positions of the media-exine boundary peaks.

Therefore, the media-exine boundary peaks (the front wall side media-exine boundary peak and the rear wall side media-exine boundary peak) are detected out of measurement data of a reflected wave obtained at certain time. The media-exine boundary peaks can be detected by performing, for example, processing for comparing measurement data with a predetermined threshold or processing for calculating differential of a value and comparing the differential with a threshold. Fluctuation in a blood vessel diameter is detected from phase changes of the reflected wave from depths corresponding to the detected media-exine boundary peaks.

FIG. 3A shows fluctuation in a blood vessel diameter in one heartbeat period among the blood vessel diameter fluctuations detected as explained above. In FIG. 3A, the abscissa indicates time and the ordinate indicates a blood vessel diameter. Each plot indicates a sample timing for a blood vessel diameter. At each sample timing, measurement data of the reflected wave shown in FIG. 2A is obtained.

The fluctuation in the blood vessel diameter in one heartbeat period shows tendency substantially the same as fluctuation in a blood pressure in one heartbeat period. The blood pressure rises when an ejection wave is sent from the heart according to opening of the aortic valve. The blood vessel diameter also increases according to the rise in the blood pressure. A blood vessel diameter A1 at time t1 is a blood vessel diameter (a diastole blood vessel diameter) corresponding to a minimum blood pressure (a diastole blood pressure).

Blood is ejected from the heart according to opening of the aortic valve. The blood vessel diameter steely rises from the diastole blood vessel diameter A1. At time t2, a peak E1 of the ejection wave is observed. Thereafter, the blood vessel diameter slightly decreases and then increases again. A peak T1 of a tidal wave is observed at time t3 because of the influence of the tidal wave, which is a reflected wave from an artery branch section.

Thereafter, the blood vessel diameter decreases and, according to closing of the aortic valve, a notch N1 is observed at time t. The notch N1 is equivalent to the end of the systole. Thereafter, a blood flow rushes to the aortic valve with the aortic pressure. As a result, a dicrotic wave, which is a reflected vibration wave, occurs. Consequently, the blood vessel diameter temporarily increases. A peak D1 of the dicrotic wave is observed at time t5. Thereafter, the blood vessel diameter gently decreases. At time t6, the blood vessel diameter reaches a diastole blood vessel diameter A2 of the next beat at time t6.

According to a general definition, a period from opening of the aortic valve to closing of the aortic valve is the “systole” and a period from closing of the aortic valve to opening of the next aortic valve is the “diastole”. Therefore, in FIG. 3A, the systole and the diastole are shown to correspond to the fluctuation in the blood vessel diameter. One heartbeat period is formed by the systole and the diastole.

### 2.2 Combination of Measurement Data

A blood vessel diameter stable period in which the blood vessel diameter is in a stable state in one heartbeat period is determined on the basis of the fluctuation in the blood vessel diameter detected as explained above by a processing unit 100 in the main body apparatus 20 described later. In the blood vessel diameter stable period, since there is almost no difference in the blood vessel diameter, there is almost no change in a position of the blood vessel wall from the body surface. Measurement data (data shown in FIG. 2A) of a reflected wave at sample timings included in this period are similar data. Therefore, measurement data measured in the blood vessel diameter stable period are combined.

This embodiment is focused on the end of the diastole (hereinafter referred to as “diastole end”). A portion P1 surrounded by a dotted line in FIG. 3A is the diastole end. In this period, a change in the blood vessel diameter is very small. Therefore, for example, tracing back from sample timing when the blood vessel diameter is the smallest, i.e., sample timing when a diastole blood vessel diameter (a minimum blood vessel diameter) is obtained, a period in which a fluctuation amount in the blood vessel diameter from the diastole blood vessel diameter, for example, a difference of the maximum of the diastole blood vessel diameter which are actually measured by an ultrasonic probe 10 described later from an arithmetic mean or a geometric mean of the diastole blood vessel diameter which are actually measured by the ultrasonic probe 10 described later is equal to or smaller than a predetermined threshold (e.g., 10 μm) is determined by the processing unit 150 in the main body apparatus 20 described later. The determined period is set as a blood vessel diameter stable period by an adding and averaging unit 150 in the main body apparatus 20 described later. Measurement data in the blood vessel diameter stable period is combined. Specifically, the measurement data is added up and averaged at sampling timings in the blood vessel diameter stable period.

FIG. 2B is a diagram showing an example of combined data obtained by adding up and averaging measurement data in the diastole end. When the combined data shown in FIG. 2B and the measurement data shown in FIG. 2A are compared, it is seen that a lumen-intima boundary peak is clear in the combined data compared within the measurement data.

In the measurement data shown in FIG. 2A, a lot of noise having amplitude in the same degree as the rear wall side lumen-intima boundary peak is observed in a region of depths d10 to d20. However, in the combined data shown in FIG. 2B, the amplitude of the noise is small. This is because, since the noise observed at the depth d10 to d20 is random noise, the noise is averaged by the combination and the amplitude of the noise is close to zero. This is more conspicuous as a frame rate is increased and the number of measurement data to be added up and averaged is increased. Consequently, the lumen-intima boundary peak becomes relatively clear compared with a noise component and it is easy to detect a peak.

In the peak detection, concerning each of the front wall side and the rear wall side, a depth range in which the lumen-intima boundary can be present is set as a search range and peak search is performed in the search range to detect the lumen-intima boundary peak by a peak detecting unit 160 in the main body apparatus 20 described later. The blood vessel diameter is substantially minimum in the diastole end. However, it is possible to estimate in advance a degree of the thickness of the blood vessel on the basis of physiological knowledge and measurement. The blood vessel diameter is known as explained with reference to FIGS. 3A and 35. Therefore, the lumen-intima boundary can be present around a position on the inner side by the length equivalent to the thickness from the external shape of the blood vessel diameter. Instead of the length, a ratio of the thickness to the blood vessel diameter can be used.

Consequently, a search range in which the lumen-intima boundary peak is searched can be set for each of the front wall side and the rear wall side according to the diastole end. The peak search is performed in the search range to detect the lumen-intima boundary peak.

In FIG. 2B, the search range on the front wall side (hereinafter referred to, as “front wall side search range”) and the search range on the rear wall side (hereinafter referred to as “rear wall side search range”) are schematically shown on a graph. In the peak search, in the search ranges, for example, threshold determination for the amplitude of a reflected wave is performed to determine maximum amplitude among amplitudes exceeding the threshold as the lumen-intima boundary peak. It is also possible that a differential value of amplitude is calculated and threshold determination is performed for the differential value to detect a peak.

In FIG. 2B, a peak Pc having height of a degree same as the rear wall side lumen-intima boundary peak appears at depth d15 between the depth d10 and the depth d20. This is caused by the influence of multiple reflection in the lumen of the measurement target blood vessel. The peak Pc appears to be the front wall side lumen-intima boundary peak. However, the depth of the peak Pc is outside the front wall side search range. Therefore, it can be determined that the peak Pc is not the lumen-intima boundary peak.

### 2-3. Calculation of a Blood Vessel Inner Diameter

If the lumen-intima boundary peak can be detected as explained above, a blood vessel inner diameter is calculated from a difference between depth corresponding to the front wall side lumen-intima boundary peak and depth corresponding to the rear wall side lumen-intima boundary peak by a blood-vessel diameter fluctuation calculating unit 170 in the main body apparatus 20 described later. An instantaneous value of the blood vessel inner diameter can be calculated in this way.

Depending on a use, fluctuation in the blood vessel inner diameter can also be calculated by the blood-vessel diameter fluctuation calculating unit 170 in the main body apparatus 20 described later. For example, when a use for estimating a blood pressure using the blood vessel inner diameter is assumed, it is necessary to calculate fluctuation in the blood vessel inner diameter in order to estimate fluctuation in a blood pressure. In this case, a blood vessel diameter only has to be continuously calculated using, for example, a phase difference tracking method.

Specifically, concerning each of the front wall side and the rear wall side, a predetermined depth range centering on the lumen-intima boundary peak detected as explained above is designated as a tracking range. A phase of a reflected wave is tracked in the tracking range to continuously calculate the blood vessel inner diameter. This is equivalent to processing for setting as a tracking target a position in measurement data of a reflected wave peak from the lumen-intima boundary, tracking the position of the reflected wave peak in the continuous measurement data, and calculating fluctuation in the blood vessel inner diameter.

3. Functional configuration

FIG. 4 is a block diagram showing an example of a functional configuration of the ultrasonic measuring apparatus 1. The ultrasonic measuring apparatus 1 includes the ultrasonic probe 10 and the main body apparatus 20.

The ultrasonic probe 10 is a small contactor configured to switch, according to a control signal from a processing unit 100, a transmission mode and a reception mode of ultrasound in a time division manner and transmit and receive the ultrasound. A reception signal of reflected wave of the ultrasound is output to the processing unit 100.

The main body apparatus 20 includes the processing unit 100, an operation unit 200, a display unit 300, a sound output unit 400, a communication unit 500, a clock unit 600, and a storing unit 800.

The processing unit 100 is a control device and a calculating device configured to comprehensively control the units of the ultrasonic measuring apparatus 1. The processing unit 100 includes a microprocessor such as a CPU (Central Processing Unit) or a DSP (Digital Signal Processor) and an ASIC (Application Specific Integrated Circuit).

The processing unit 100 includes, as main functional units, a reflected-wave measuring unit 120, a diameter-fluctuation detecting unit 130, an addition/averaging-period setting unit 140, an adding and averaging unit 150, a peak detecting unit 160, a blood-vessel-inner-diameter calculating unit 170, and a blood-vessel-inner-diameter-fluctuation calculating unit 180. The functional units are described as examples only. The processing unit 100 does not always have to include all the functional units as essential components. Naturally, the processing unit 100 may include other functional units as essential components.

The reflected-wave measuring unit 120 calculates, on the basis of a reception signal of a reflected wave output from the ultrasonic probe 10, for example, reflected wave measurement data 820 obtained by calculating the amplitude of the reflected wave per depth.

The diameter-fluctuation detecting unit 130 detects fluctuation in an inter-media-exine boundary distance as the diameter fluctuation using the reflected wave measurement data 820 for each measurement timing (each frame) calculated by the reflected-wave measuring unit 120.

The addition/averaging-period setting unit 140 sets, on the basis of the diameter fluctuation detected by the diameter-fluctuation detecting unit 130, a period in which the adding and averaging unit 150 performs addition and averaging (hereinafter referred to as “addition and averaging period”). The addition/averaging-period setting unit 140 is equivalent to a determining unit configured to determine, on the basis of a result of the detection of the diameter fluctuation, a blood vessel diameter stable period in which a blood vessel diameter is in a stable state in one heartbeat period.

The adding and averaging unit 150 subjects the reflected wave measurement data 820 to addition and averaging processing at measurement timing in the addition and averaging period set by the addition/averaging-period setting unit 140. The adding and averaging unit 150 is equivalent to a combining unit configured to combine measurement data measured in the blood vessel diameter stable period.

The peak detecting unit 160 detects, out of combined data 830 calculated by the adding and averting unit 150, lumen-intima boundary peaks (a front wall side lumen-intima boundary peak and a rear wall side lumen-intima boundary peak) according to the principle explained above.

The blood-vessel-inner-diameter calculating unit 170 calculates a blood vessel inner diameter from a difference between depths equivalent to the lumen-intima boundary peaks detected by the peak detecting unit 160.

The blood-vessel-inner-diameter-fluctuation calculating unit 180 sets, as a reference value, the blood vessel inner diameter calculated by the blood-vessel-inner-diameter calculating unit 170 and calculates fluctuation in the blood vessel inner diameter using, for example, a phase difference tracking method.

The operation unit 200 is an input device including button switches and the like. The operation unit 200 outputs a signal of a depressed button to the processing unit 100. According to the operation of the operation unit 200, various instruction inputs such as a measurement start instruction for a blood vessel inner diameter are input. The operation unit 200 is equivalent to the operation buttons 24 shown in FIG. 1A.

The display unit 300 is a display device including an LCD (Liquid Crystal Display) and configured to perform various kinds of display based on display signals input from the processing unit 100. On the display unit 300, information concerning the blood vessel inner diameter and the like calculated by the blood-vessel-inner-diameter calculating unit 170 and the blood-vessel-inner-diameter-fluctuation calculating 180 are displayed. The display unit 300 is equivalent to the liquid crystal display device 25 shown in FIG. 1A.

The sound output unit 400 is a sound output device configured to perform various sound outputs based on sound output signals input from the processing unit 100. For example, the sound output unit 400 outputs alarm sounds for measurement start, measurement end, error occurrence, and the like. The sound output unit 400 is equivalent to the speaker 26 shown in FIG. 1A,

The communication unit 500 is a communication device for transmitting and receiving, according to the control by the processing unit 100, information to be used in the inside of the apparatus between the communication unit 500 and an external information processing apparatus. As a communication system of the communication unit 500, it is possible to apply various systems such as a mode for wire-connecting the communication unit 500 via a cable conforming to a predetermined communication standard, a mode for connecting the communication unit 500 to a charger called cradle via a shared intermediate apparatus, and a mode for wireless-connecting the communication unit 500 using short-range distance radio communication.

The clock unit 600 is a timing device including a crystal oscillator, which is configured by a crystal resonator and an oscillation circuit, and configured to measure time. measured time of the clock unit 600 is output to the processing unit 100 at any time.

The storing unit 800 includes storage devices such as a ROM (Read Only Memory), a flash ROM, and a RAM (Random Access Memory). The storing unit 800 has stored therein a system program of the ultrasonic measuring apparatus 1, various programs for realizing various functions such as a blood vessel inner diameter measuring function, data, and the like. The storing unit 800 includes a work area for temporarily storing data being subjected to various kinds of processing, a processing result, and the like.

The storing unit 800 has stored therein, as a program, for example, a blood vessel inner diameter measuring program 810 to be read out by the processing unit 100 and executed as blood vessel inner diameter measurement processing (see FIG. 7). This processing is explained in detail below with reference to a flowchart.

The storing unit 800 has also stored therein, as data, reflected wave measurement data 820, combined data 830, diameter fluctuation detection data 840, reference measurement data 850, and blood vessel inner diameter measurement data 860.

The reflected wave measurement data 820 is measurement data of a reflected wave measured by the reflected-wave measuring unit 120 and is measurement data indicating, for example, a relation between depth and the amplitude of the reflected wave. For example, the data shown in FIG. 2A is equivalent to the reflected wave measurement data 820. One measurement data is equivalent to data (frame data) for one frame.

The combined data 830 is data obtained by the adding and averaging unit 150 adding up and averaging the reflected wave measurement data 820. For example, the data shown in FIG. 2B is equivalent to the combined data 830.

The diameter fluctuation detection data 840 is data of diameter fluctuation detected by the diameter-fluctuation detecting unit 130 on the basis of measurement data continuing in time series. The data shown in FIGS. 3A and 3B is equivalent to the diameter fluctuation detection data 840.

The reference measurement data 850 is data serving as a reference for performing blood vessel inner diameter measurement. A data configuration example of the reference measurement data 850 is shown in FIG. 5. In the reference measurement data 850, an addition and averaging period 850A, peak depth 850B, and a reference blood vessel inner diameter 850C are stored in association with one another.

The addition and averaging period 850A is an addition and averaging period set by the addition/averaging period setting unit 140. An addition and averaging period set on the basis of time associated with the diameter fluctuation detection data 840 is stored in the addition and averaging period 850A.

The peak depth 850B is depth corresponding to the lumen-intima boundary peak. Peak depth is stored concerning each of the front wall side and the rear wall side.

The reference blood vessel inner diameter 850C is a blood vessel inner diameter calculated from a difference in the peak depth 850B. The blood vessel inner diameter is a reference value of the blood vessel inner diameter.

The blood vessel inner diameter measurement data 860 is data in which a measurement result of the blood vessel inner diameter is stored. A data configuration example of the blood vessel inner diameter measurement data 860 is shown in FIG. 6. In the blood vessel inner diameter measurement data 860, a blood vessel inner diameter 860B continuously measured using the phase difference tracking method is stored in time series in association with measurement time 860A.

### 4. Flow of Processing

FIG. 7 is a flowchart for explaining a flow of blood vessel inner diameter measurement processing executed by the processing unit 100 according to the blood vessel inner diameter measuring program 810 stored in the storing unit 800.

The processing unit 100 controls the ultrasonic probe 10 to start transmission and reception of ultrasound (step A1). The reflected-wave measuring unit 120 starts measurement of a reflected wave on the basis of a reception signal of a reflected wave of the ultrasound and causes the storing unit 800 to store data of the measurement as the reflected wave measurement data 820.

Subsequently, the diameter-fluctuation detecting unit 130 performs diameter fluctuation detection processing (step A5). Specifically, the diameter-fluctuation detecting unit 130 detects media-exine boundary peaks (a front wall side media-exine boundary peak and a rear wall side media-exine boundary peak) out of latest data in the reflected wave measurement data 820 stored in the storing unit 800. The diameter-fluctuation detecting unit 130 performs predetermined diameter fluctuation analysis processing on the basis of a phase change of the reflected wave from depths equivalent to the detected media-exine boundary peaks and analyzes fluctuation in the blood vessel diameter. The diameter-fluctuation detecting unit 130 causes the storing unit 800 to store a result of the analysis as the diameter fluctuation detection data 840.

Thereafter, the addition/averaging-period setting unit 140 sets an addition and averaging period (step A7). Specifically, the addition/averaging-period setting unit 140 determines, on the basis of the diameter fluctuation detected by the diameter-fluctuation detecting unit 130, a period in which the diameter fluctuation is equal to or smaller than a predetermined threshold (e.g., 10 μm) tracing back from timing when the blood vessel diameter is the smallest in one heartbeat period and sets the period as the addition and averting period.

Subsequently, the adding and averaging unit 150 adds up and averages the reflected wave measurement data 820 at measurement timings included in the addition and averaging period and causes the storing unit 800 to store a result of the addition and averaging as the combined data 830.

Subsequently, the peak detecting unit 160 performs predetermined threshold determination in predetermined search ranges (a front wall side search range and a rear wall side search range) concerning each of the front wall side and the rear wall side and detects lumen-intima boundary peaks out of the combined data 830 (step A11). The blood-vessel-inner-diameter calculating unit 170 calculates a reference value of the blood vessel inner diameter from a difference between depths of the lumen-intima boundary peaks and causes the storing unit 800 to store a result of the calculation in the reference measurement data 850 (step A13).

Thereafter, the blood-vessel-inner-diameter-fluctuation calculating unit 180 sets a phase difference tracking range having predetermined width centering on the depths corresponding to the lumen-intima boundary peaks detected in step A11. The blood-vessel-inner-diameter-fluctuation calculating unit 180 tracks a phase of the reflected wave within the phase difference tracking range to calculate fluctuation in the blood vessel inner diameter and causes the storing unit 800 to store a result of the calculation in the blood vessel inner diameter measurement data 860 (step A15).

Subsequently, the blood-vessel-inner-diameter calculating unit 170 determines whether output timing for the blood vessel inner diameter comes (step A17). When determining that the output timing does not come (No in step A17), the blood-vessel-inner-diameter calculating unit 170 shifts to step A21. When determining that the output timing comes (Yes in step A17), the blood-vessel-inner-diameter calculating unit 170 performs control for causing the display unit 300 to display the latest blood vessel inner diameter (step A19).

Thereafter, the processing unit 100 determines whether to end the processing (step A21). For example, the processing unit 100 determines whether instruction operation for the end of the measurement of the blood vessel inner diameter is performed by the subject via the operation unit 200. When determining to continue the processing (No instep A21), the processing unit 100 returns to step A17. When determining to end the processing (Yes in step A21), the processing unit 100 ends the blood vessel inner diameter measurement processing.

### 5. Action and Effects

In the ultrasonic measuring apparatus 1, the diameter-fluctuation detecting unit 130 repeatedly executes emission of ultrasound from the ultrasonic probe 10 and measurement of a reflected wave from the blood vessel and detects fluctuation in a blood vessel diameter using measurement data. The addition/averaging-period setting unit 140 determines, on the basis of a detection result of fluctuation in the blood vessel diameter, a blood vessel diameter stable period in a state in which the blood vessel diameter is stable in one heartbeat period and sets the blood vessel diameter stable period as an addition and averaging period. The adding and averaging unit 150 adds up and averages measurement data measured within the addition and averaging period. The blood-vessel-inner-diameter calculating unit 170 calculates a blood vessel inner diameter of the blood using data combined by the combining unit.

The measurement data in the period in which the blood vessel diameter is a stable state in one heartbeat period is added up and averaged. Consequently, it is possible to attenuate noise component and highly accurately detect the lumen-intima boundary peaks. In particular, in this embodiment, a diastole end in a diastole period is set as a blood vessel diameter stable period and measurement data in the diastole end is combined. In the diastole end in one heartbeat period, the blood vessel diameter is in a stable state. Measurement data of the reflected wave obtained in this period is data similar to one another. Therefore, it is possible to cause the lumen-intima boundary peaks to appear by superimposing the measurement data.

A period suitable for the addition and averaging is examined. For example, it is assumed that an ultrasonic signal having a frequency of 7.5 MHz is repeatedly transmitted and received. When interference of waves is taken into account, within time until reflected waves from the same region deviate by a quarter wavelength (25.5 μm in terms of distance: 1530 m/s in terms of sound speed), the reflected waves can be intensified one another by superimposition of the reflected waves. Since the quarter wavelength is 25.5 μm in terms of distance, in a period in which fluctuation in the blood vessel diameter is equal to or smaller than 25.5 μm, it is possible to improve an SN (Signal Noise) ratio by adding up and averaging measurement data in the period. In this embodiment, a threshold of a fluctuation amount of the blood vessel diameter in determining the blood vessel diameter stable period is set to 10 μm, which is shorter than the quarter wavelength. Therefore, it is possible to sufficiently expect improvement of the SN ratio.

### 6. Modification

An embodiment applicable with the invention is not limited to the embodiment explained above. It goes without saying that the embodiment can be changed as appropriate without departing from the spirit of the invention. A modification is explained below.

### 6-1. Measurement Target Blood Vessel

In the example explained in the embodiment, the carotid is set as the measurement target blood vessel and the blood vessel inner diameter of the carotid is measured. However, the measurement target vessel is not limited to this. Besides, for example, the artery of the limbs such as the radial artery or the brachial artery may be set as the measurement target blood vessel.

### 6-2. Ultrasonic Measuring Apparatus

In the embodiment, the ultrasonic measuring apparatus for measuring a blood vessel inner diameter is illustrated and explained as the measuring apparatus hung from the neck of the subject and used. However, this configuration is only an example. Besides, for example, a main body apparatus wound around the upper arm of the subject and used may be configured or a main body apparatus worn on the wrist of the subject and used may be configured. The ultrasonic probe and the main body apparatus do not always have to be separate. A measuring apparatus in which the ultrasonic probe and the main body apparatus are provided in the same housing may be configured.

The embodiment is explained as an embodiment of the measuring apparatus used by the subject, who is taking free action, to personally measure a blood vessel inner diameter. However, an application range of the invention is not limited to this. The invention can be applied to an ultrasonic measuring apparatus for medical use, for example, an ultrasonic testing apparatus with which a technician applies an ultrasonic test to a lying subject using an ultrasonic probe.

The ultrasonic measuring apparatus for measuring a blood vessel inner diameter in the embodiment may be provided in a blood pressure measuring apparatus for measuring a blood pressure. The blood vessel inner diameter and the blood pressure can be associated with each other according to a linear or nonlinear publicly-known correlation characteristic. That is, it is possible to estimate the blood pressure from the blood vessel inner diameter according to a publicly-known arithmetic expression including the block vessel inner diameter as a variable.

### 6-3. Addition and Averaging Period

In the explanation in the embodiment, the diastole end is detected from one heartbeat period and set as the addition and averaging period. However, this is only an example. In the diastole, besides the diastole end, there is a period in which the blood vessel diameter is in a stable state. Therefore, the blood vessel diameter stable period only has to be determined from the diastole. A target period of addition and averaging does not have to be the diastole end.

FIG. 8 shows a graph of blood vessel diameter fluctuation same as FIG. 2A. In the figure, for example, in periods equivalent to a portion P2 and a portion P3 surrounded by dotted lines in a diastole middle period, the blood vessel diameter is in a stable state. Therefore, these portions may be detected out of blood vessel diameter fluctuation and set as the addition and averaging period.

The position of the blood vessel wall from the body surface fluctuates in one heartbeat period. Therefore, the search range of the lumen-intima boundary peaks explained with reference to FIG. 2B could be different according to which range in one heartbeat period is set as the addition and averaging period. Therefore, it is effective to variably set the search range according to the addition and averaging period. In this case, in the ultrasonic measuring apparatus 1, a search-range setting unit is configured as a functional unit of the processing unit 100. The search-range setting unit sets a search range corresponding to the addition and averaging period set by the addition/averaging-period setting unit 140.

FIG. 9 is a flowchart showing a part of steps extracted from a processing flow of second blood vessel inner diameter measurement processing executed by the processing unit 100 of the ultrasonic measuring apparatus 1 in the embodiment in this modification. The second blood vessel inner diameter measurement processing is processing substantially the same as the blood vessel inner diameter measurement processing shown in FIG. 7. Three steps shown in FIG. 9 are added between steps A5 and A9 of the blood vessel inner diameter measurement processing.

After performing the diameter fluctuation calculation processing in step A5 in FIG. 7, the addition/averaging-period setting unit 140 extracts a period in which fluctuation in the blood vessel diameter satisfies a predetermined stabilization condition (step B1). Specifically, for example, a difference of the maximum of the diastole blood vessel diameter which are actually measured from an arithmetic mean or a geometric mean of the diastole blood vessel diameter which are actually measured is equal to or smaller than a predetermined threshold (e.g., 10 μm) is extracted out of the blood vessel fluctuation obtained as shown in FIG. 3A.

Subsequently, the addition/averaging-period setting unit 140 selects a period with a largest number of samples in the period extracted in step B1 and sets the period as the addition and averaging period (step B3). The period with the largest number of samples is selected because, when the number of samples is larger, it is possible to more effectively attenuate noise when measurement data is combined.

Thereafter, the search-range setting unit sets a search range on the basis of depth corresponding to the period selected in step B3 (step B5). Specifically, the search-range setting unit determines the time in the center of the period selected in step B3. The search-range setting unit refers to the reflected wave measurement data 820 corresponding to the time in the center and determines the depth corresponding to a media-exine boundary peak concerning each of the front wall side and the rear wall side. If the depth corresponding to the media-exine boundary peak is known, it is possible to estimate, on the basis of information concerning thickness and the like of the membranes (the exine, the media, and the intima) forming the blood vessel, a rough depth range (i.e., search range) in which the lumen-intima boundary peak is present.

### 6-4. Combination of Measurement Data

Effects same as the effects in the embodiment can be obtained by combining data of a waveform obtained by full-wave rectifying the reflected wave or data of a waveform obtained by subjecting the reflected wave to logarithmic compression rather than adding up and averaging the measurement data of the reflected wave itself.

The entire disclosure of Japanese Patent Application No. 2012-173751, filed on Aug. 6, 2012 and No. 2013-161074, filed on Aug. 2, 2013 are expressly incorporated by reference herein.

What is claimed is:

1. An ultrasonic measuring apparatus that emits ultrasound to a blood vessel, executes measurement of a reflected wave from the blood vessel, and calculates a blood vessel diameter using measurement data, the ultrasonic measurement apparatus comprising:
a combining unit configured to combine a plurality of the measurement data measured in a blood vessel diameter stable period in which a fluctuation amount of the blood vessel diameter is equal to or smaller than a threshold in a heartbeat period; and
a blood-vessel-diameter calculating unit configured to calculate the blood vessel diameter using the data combined by the combining unit.

2. The ultrasonic measuring apparatus according to claim 1, wherein the combining unit adds up and averages the measurement data measured in the blood vessel diameter stable period.

3. The ultrasonic measuring apparatus according to claim 1, further comprising a determining unit configured to determine the blood vessel diameter stable period on the basis of the measurement data, wherein the determining unit determine the blood vessel diameter stable period from a diastole.

4. The ultrasonic measuring apparatus according to claim 1, wherein the blood-vessel-diameter calculating unit includes a peak detecting unit configured to detect a reflected wave peak of a boundary between a lumen and an intima of the blood vessel from the combined data and calculates the blood vessel diameter on the basis of the reflected wave peak.

5. The ultrasonic measuring apparatus according to claim 4, further comprising a range setting unit configured to set, on the basis of the measurement data, a depth range in which the boundary between the lumen and the intima of the blood vessel can be present in the blood vessel diameter stable period, wherein
the peak detecting unit detects the reflected wave peak using the depth range.

6. The ultrasonic measuring apparatus according to claim 4, further comprising a blood-vessel-diameter-fluctuation calculating unit configured to set a position in the measurement data of the reflected wave peak detected by the peak detecting unit as a tracking target, track the position of the reflected wave peak in the continuous measurement data, and calculate fluctuation in the blood vessel diameter.

7. A blood vessel diameter calculating method by an ultrasonic measuring apparatus that executes emission of ultrasound and measurement of a reflected wave from a blood vessel and detects fluctuation in a blood vessel diameter using measurement data, the blood vessel diameter calculating method comprising:
combining a plurality of the measurement data measured in a blood vessel diameter stable period in which a fluctuation amount of the blood vessel diameter is equal to or smaller than a threshold in a heartbeat period; and
calculating the blood vessel diameter using the combined data.
